(19) United States
(12) Patent Application Publication
(10) Pub. No.: US 2014/0004072 A1
(43) Pub. Date: Jan. 2, 2014

(54) Polyethyleneimine And Beta2-Adrenergic Receptor Based Gene Therapy For Acute Lung Injury

(71) Applicants: Cheng-Wen Wu, Taipei (TW); Erh-Hsuan Lin, Taipei (TW); Hsiang-Yi Chang, Taipei (TW)

(72) Inventors: Cheng-Wen Wu, Taipei (TW); Erh-Hsuan Lin, Taipei (TW); Hsiang-Yi Chang, Taipei (TW)

(73) Assignee: NATIONAL YANG MING UNIVERSITY, Taipei (TW)

(21) Appl. No.: 13/539,300

(22) Filed: Jun. 29, 2012

(51) Int. Cl. A61K 48/00; C08G 73/04; A61P 11/00

(52) U.S. Cl. 424/78.3; 525/54.2

(57) ABSTRACT

A method and a composition of gene therapy for treating acute lung injury (ALI) and acute respiratory distress syndrome (ARDS) based on polyplexes formed between linear polyethyleneimine (PEI) and DNA comprising the β2-Adrenergic Receptor (β2AR) gene are provided.

### TECHNICAL FIELD

The present disclosure relates to gene therapy for treating acute lung injury (ALI) and acute respiratory distress syndrome (ARDS). More specifically, the present disclosure relates to using linear polyethyleneimine (PEI) as a non-viral gene delivery vector to deliver deoxyribonucleic acid (DNA) comprising β2-Adrenergic Receptor (β2AR) genes for ALI/ARDS therapy.

### BACKGROUND

Acute lung injury (ALI) and its more severe form, acute respiratory distress syndrome (ARDS), are devastating clinical syndromes. They are associated with inflammatory injury to lung epithelium/endothelium and the passage of protein-rich edema into air spaces, leading to noncompliant lungs that function poorly in gas exchange. Over the past decades, the progress made in treating ALI/ARDS is only moderate, and the mortality remains as high as 30-50%. Current clinical recovery depends mainly on the use of lung-protective ventilation with low tidal volumes. Although a number of promising pharmacologic therapies, including β2-Adrenergic Receptor (β2AR) agonists, have been evaluated in Phase II/III clinical trials for the treatment of ALI/ARDS, unfortunately, no substantial improvements in reducing mortality in ALI/ARDS have been achieved so far.

Gene therapy is a promising approach for treating a variety of chronic and acute diseases. However, gene therapy has not been widely adopted for treating ALI/ARDS. This may be due to the absence of suitable delivery vectors because ALI/ARDS are associated with acute inflammatory injuries in alveolar epithelia and usually life-threatening, gene therapy applied on patients suffering ALI/ARDS must cause no or less inflammatory. Both viral and non-viral vectors that are commonly used in gene therapy have serious drawbacks that limit their efficacies in treating ALI/ARDS. Ideally, vectors should be safe, can deliver genes in a rapid, efficient, and transient manner. In addition, they should mainly target on pathologic loci. Viral vectors are typically more efficient in gene delivery than non-viral vectors, but they bear the risk of mutational insertions, carcinogenesis, and the induction of strong inflammatory responses. Non-viral vectors are more attractive because they are relatively safe, causing less inflammation, and capable of transferring larger genes. However their relatively low delivery efficiency and poor transgene expression hamper their use in the clinics.

Recently, a feasible gene therapy in an animal model of ALI was reported, in which the Na⁺, K⁺-ATPase genes were delivered to the lungs of mice by electroporation. Improvements in alveolar fluid clearance (AFC) and respiratory mechanics were observed after genes were delivered to the lungs of mice with pre-existing ALI induced by lipopolysaccharide (LPS). However, this method was not able to ameliorate the syndrome or improve the survival when a severe ALI was induced by high-dose LPS. Therefore, although the delivery of therapeutic genes might be an effective and logical approach to treat ALI, its application in sever lung injury still needs refinement.

β2AR is a G protein-coupled receptor presenting throughout the lung. Activation of β2AR can regulate important factors that are critical for alveolar ion and fluid transport, thus decreasing neutrophil-related inflammation and improving alveolar epithelial repair. Therefore, β2AR signaling has gained considerable interest in ALI/ARDS therapy because of its ability to improve the resolution of pulmonary edema. It has been shown that overexpression of β2AR in mice can increase AFC and protect mice from later induced ALI. These research models, however, are not applicable in gene therapy for pre-existing ALI/ARDS because they use transgenic mice or adenovirus for gene delivery that can further induce strong inflammatory responses. In fact, no consistent results have been obtained from clinic trials using β2AR agonist to treat pre-existing ALI/ARDS. This could be attributed to the adverse side effect of β2AR agonist. β2AR agonist can cause an increase in cardiac contractility as well as an increase in cardiac output, thus leading to increased lung endothelial permeability. As a result, pulmonary edema is aggravated. Therefore, there remains a need to develop a safe and efficient method to deliver β2AR genes to lungs for treating pre-existing ALI/ARDS.

### SUMMARY

The present disclosure provides a non-viral gene delivery method and composition for treating acute lung injury (ALI) and acute respiratory distress syndrome (ARDS) by using polyplexes formed between polyethyleneimine (PEI) and deoxyribonucleic acid (DNA) comprising β2-Adrenergic Receptor (β2AR) genes.

In one aspect, methods of delivering the β2AR genes to a lung of a patient in need thereof to treat a lung disease in the patient are provided.

In one embodiment, a method of delivering the β2AR genes to a lung of a patient in need thereof to treat a lung disease in the patient may comprise administering to the patient a polyplex comprising a linear PEI and a DNA comprising the β2AR gene.

In some embodiments, the lung disease may be acute lung injury and acute respiratory distress syndrome.

In some embodiments, the linear PEI has a molecular weight of about 22,000 to about 25,000 daltons.

In some embodiments, the molar ratio of the nitrogen atoms in the linear PEI to phosphates in the DNA comprising the β2AR gene may be about 6 to about 10.

In some embodiments, the weight ratio of the DNA comprising the β2AR gene to the patient may be less than 2.5×10⁻⁶.

In some embodiments, the administering may comprise administering via intravenous injection.

In another embodiment, a method of delivering β2AR genes into alveolar epithelial cells of a lung of a patient in need thereof to treat acute lung injury in the patient may comprise mixing a linear PEI with a DNA comprising the β2AR gene to form a polyplex, and administering the polyplex to alveolar epithelial cells of the lung via intravenous injection.

In some embodiments, the linear PEI may have a molecular weight of about 22,000 to about 25,000 daltons.

In some embodiments, the molar ratio of the nitrogen atoms in the linear PEI to DNA phosphates in the DNA comprising the β2AR gene may be about 6 to about 10.

In some embodiments, the weight ratio of the DNA comprising the β2AR gene to the patient may be less than 2.5×10⁻⁶.

In still another aspect, a polyplex for delivering β2AR genes to a lung of a patient in need thereof to treat a lung disease in the patient may comprise a linear PEI and a DNA comprising the β2AR gene.

In some embodiments, the linear PEI may have a molecular weight of about 22,000 to about 25,000 daltons.

In some embodiments, the molar ratio of the nitrogen atoms in the linear PEI to DNA phosphates in the DNA comprising the β2AR gene may be about 6 to about 10.

In some embodiments, the weight ratio of the DNA comprising the β2AR gene to the patient may be less than 2.5×10⁻⁶.

In some embodiments, the lung disease may comprise acute lung injury and acute respiratory distress syndrome.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1a-1c illustrates PEI-mediated reporter gene delivery in healthy and injured lungs. PEI/DNA nanoparticles were injected through lateral tail-vein into healthy mice or mice with pre-existing ALI. Different amounts of reporter gene expression vectors (0 to 50 μg) were complexed with 22-kD linear PEI in a constant N/P (PEI nitrogen/DNA phosphate) molar ratio of 8. Bioluminescent intensities at different time points of post-transfection were quantified (1a) (n=5 for each group, error bars indicate standard deviation), and 2 mice were illustrated as samples of imaging (1b). To determine the cell types targeted by PEI/DNA delivery, mice were sacrificed in 1 day after PEI/lacZ delivery, and lungs were removed and stained with X-Gal and haematoxylin and eosin (HE) for histochemical examination (1c). Arrows indicate the stained (blue) cells. 10 mg/kg of LPS was intratracheally instilled in mouse lung 1 hour prior to the PEI/DNA injection.

FIGS. 2a-2c illustrates exogenous and endogenous β2AR gene expressions in mice lungs with ALI. ALI was induced in mice by intratracheal instillation of 10 mg/kg of LPS. The mice were then injected with PEI/β2AR in 1 hours of post-injury (2a-2b), or not injected (2c). Mice were sacrificed at indicated time points of post-transfection, and the lungs were harvested for RNA extraction. PBS was instilled into mice lungs as non-injury control (PBS). Exogenous (human) (2a) and endogenous (mouse) (2b and 2c) β2AR gene expressions were detected using Real-Time PCR with specific primers, and presented as ratios relative to PBS group. Error bars indicate standard error of the mean (SEM). N=3 for each group. P value (2b and 2c) was calculated by two-tailed and unpaired Student's t-tests, comparing the indicated group versus PBS. *, P<0.05. **, P<0.01. ***, P<0.001.

FIGS. 3a-3e illustrates PEI/β2AR treatment improving AFC and reducing lung water content in mice with pre-existing ALI. ALI was induced in mice by intratracheal instillation of 10 mg/kg of LPS, and the gene therapy was administrated in two designs (3a). In Design 1, mice were injected with PEI/β2AR at 1 hours of post-injury and sacrificed at 24 hours; while in Design 2, mice were injected with PEI/β2AR at 24 hours of post-injury and sacrificed at 48 hours, for evaluation of therapeutic outcome. AFC was measured in vivo and shown as percentage of total instilled volume cleared in 20 minutes (3b). Lung water content was assessed by the measurement of wet-to-dry ratio (3c). The ratios of wet lung (3d) or dry lung (3e) to body weight were presented. For control, mice were treated with reporter genes (LPS+PEI/Rep, with half of luciferase and half of lacZ), or PEI alone (LPS+PEI). PBS was instilled into mice lungs as non-injury control (PBS). The PEI/DNA complex uniformly contained 30 μg of DNA with an N/P ratio of 8. Error bars indicate SEM. N=3 (3b) and n=6 (3c-3e) for each group. P value was calculated by two-tailed and unpaired Student's t-tests, comparing the indicated group versus LPS group. *, P<0.05. **, P<0.01.

FIG. 4 illustrates histopathology of injured lungs. Mice were injured by LPS and treated with PEI/β2AR following the procedures of two designs described in FIG. 3a. For control, mice were treated with reporter genes (LPS+PEI/Rep), or PEI alone (LPS+PEI). PBS was instilled into mice lungs as non-injury control (PBS). Histopathology was investigated in lung sections (5 μm, paraffin embedded) after HE staining. The lung injury score was assessed in 15 fields (×400 magnification, including a total of >300 alveoli) of lung sections from 3 mice for each condition.

FIGS. 5a-5d illustrate PEI/β2AR treatment improves BAL indexes in mice with pre-existing ALI. Mice were injured by LPS and treated with PEI/β2AR following the procedures of two designs described in FIG. 3a. The total cell numbers (5a), protein concentration (5b), and the levels of pro-inflammatory cytokines TNF-α (5c) and IL-6 (5d) in BAL were measured. For control, mice were treated with reporter genes (LPS+PEI/Rep), or PEI alone (LPS+PEI). PBS was instilled into mice lungs as non-injury control (PBS). Error bars indicate SEM. N=10 for LPS+PEI/Rep (5 mice with luciferase and 5 with lacZ), and N=5 for all the other groups. P value was calculated by two-tailed and unpaired Student's t-tests, comparing the indicated group versus LPS group. *, P<0.05. **, P<0.01. ***, P<0.001.

FIG. 6. Illustrates PEI/β2AR treatment improves the survival of mice from severe ALI. For survival assay, mice were intratracheally instilled with a high-dose LPS (40 mg/kg), and then treated with PEI/β2AR at 1 hours of post-injury. For control, mice were treated with reporter genes (LPS+PEI/Rep), or PEI alone (LPS+PEI). The survival of mice was recorded after 6 hours of post-injury. No more death was noticed after day 4. N=25 for LPS+PEI/β2AR and LPS, n=20 for LPS+PEI/Rep (10 mice with luciferase and 10 with lacZ) and LPS+PEI. P value was calculated by Gehan-Breslow statistic analysis. **, P<0.01.

### DETAILED DESCRIPTION OF SAMPLE EMBODIMENTS

The present disclosure provides a gene therapy for treating acute lung injury (ALI) and acute respiratory distress syndrome (ARDS) based on polyplexes formed between linear polyethyleneimine (PEI) and deoxyribonucleic acid (DNA) comprising 2-Adrenergic Receptors (β2AR) genes.

As used herein, “Patient” refers to any warm-blooded animal, such as human, mouse, monkey, and the like.

The terms “treat”, “treating” and “treatment” refer to the eradication or amelioration of a disease or symptoms associated with a disease.

In one aspect, methods of delivering the β2AR genes to a lung of a patient in need thereof to treat a lung disease in the patient are provided. In one embodiment, a method of delivering the β2AR genes to a lung of a patient in need thereof to treat a lung disease in the patient may comprise administering to the patient a polyplex comprising a linear PEI and a DNA comprising the β2AR gene. In another embodiment, a method of delivering β2AR genes into alveolar epithelial cells of a lung of a patient in need thereof to treat acute lung injury in the patient may comprise mixing a linear PEI with a NDA comprising the β2AR gene to form a polyplex, and administering the polyplex to alveolar epithelial cells of the lung via intravenous injection.

In another aspect, a polyplex for delivering β2AR genes to a lung of a patient in need thereof to treat a lung disease in the patient may comprise a linear PEI and a DNA comprising the β2AR gene.

Representative lung disease includes acute lung injury and acute respiratory distress syndrome.

Representative linear PEI may have a molecular weight of about 22,000 to about 25,000 daltons.

The molar ratio of nitrogen atoms in the linear PEI to phosphates in the DAN comprising the β2AR gene (N/P ratio) is preferably 6 to 10, and more preferably 8.

The weight ratio of the DNA comprising the β2AR gene to the patient is preferably less than 2.5×10-6.

The following describes the effectiveness of gene therapy for treating ALI based on polyplexes formed between linear PEI and DNA comprising the β2AR genes in a mouse model.

Polyethyleneimine (PEI) is a cationic polymer that is effective in gene delivery in vivo. It has been reported that alveolar epithelial cells (AECs) are the major target cells for PEI. PEI has also been employed as ligands immobilized on the surface of various sorbents for extracorporeal removal of endotoxin because of its high affinity to anionic LPS. However, PEI has never been employed in delivery of a therapeutic gene in vivo for ALI/ARDS treatment.

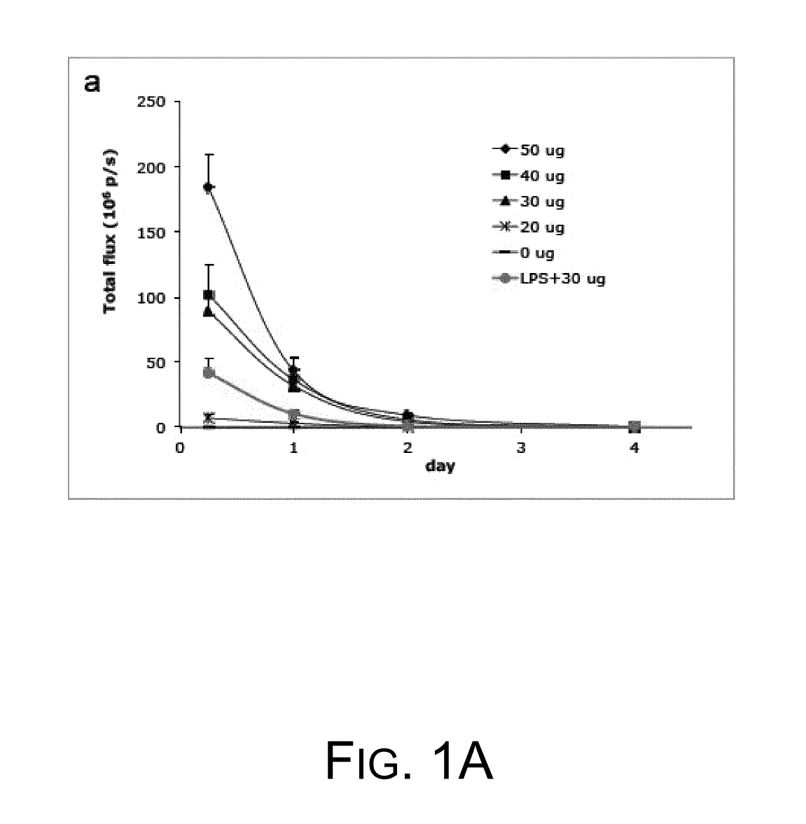

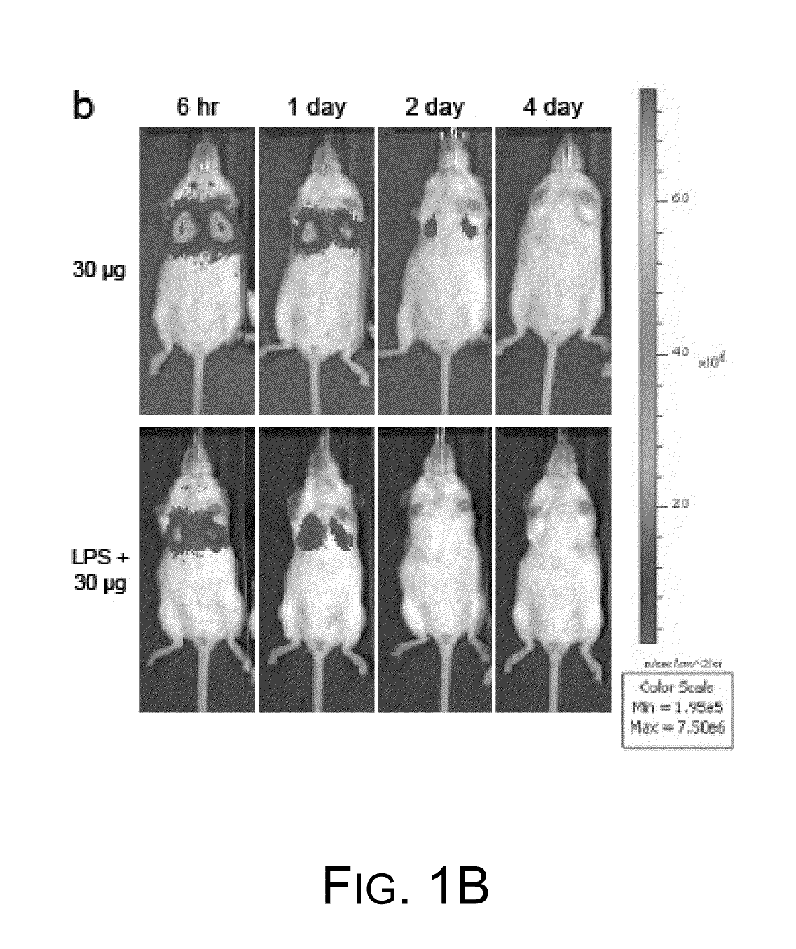

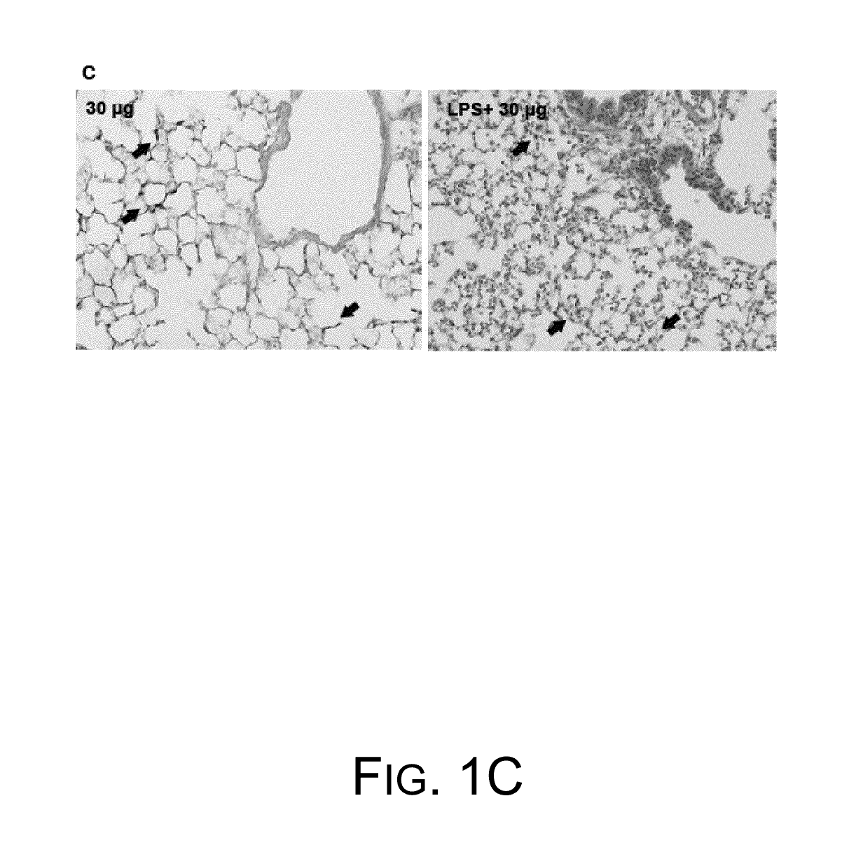

To determine whether PEI-mediated gene delivery is adaptable to ALI treatment, the kinetics and target cells of transgene expression were first analyzed. The luciferase expression in mouse lung was followed using non-invasive Bioluminescent Imaging (BLI) (FIGS. 1a-1b). It has been reported that systemic injection of PEI/DNA in mice induced an efficient gene delivery in lung, with a 100- to 1000-fold lower reporter gene signal found in other organs. The data obtained from reporter gene analysis in the present disclosure confirmed that PEI/DNA mediated a rapid, efficient, and transient transgene expression in AECs. PEI/DNA concentration reached the peak in lung as soon as 6 hours of post-injection and then declined to nearly undetectable after 4 days (FIG. 1). PEI/DNA was then applied in mice with pre-existing ALI induced by LPS. It was found that, however, delivery of a dose of 50 μg of DNA, which performed the highest transgene expression level in healthy mice, resulted in a high mortality (90%) in mice with pre-existing ALI. A lower dose of DNA (30 μg), in contrast, did not result in any death. The luciferase expression kinetics in mice with ALI was similar to that in healthy ones, although the level was reduced by around 50% (LPS+30 μg, FIGS. 1a-1b). This indicates that PEI/DNA-mediated gene delivery distributed primarily in AECs (FIG. 1c), and the pre-existing ALI did not block or alter this pattern. These data suggest that PEI could be used as a proper vector for gene delivery in ALI treatment since the gene delivery is rapid and short-lived, and targets the cells in pathologic loci despite of the presence of pre-existing ALI.

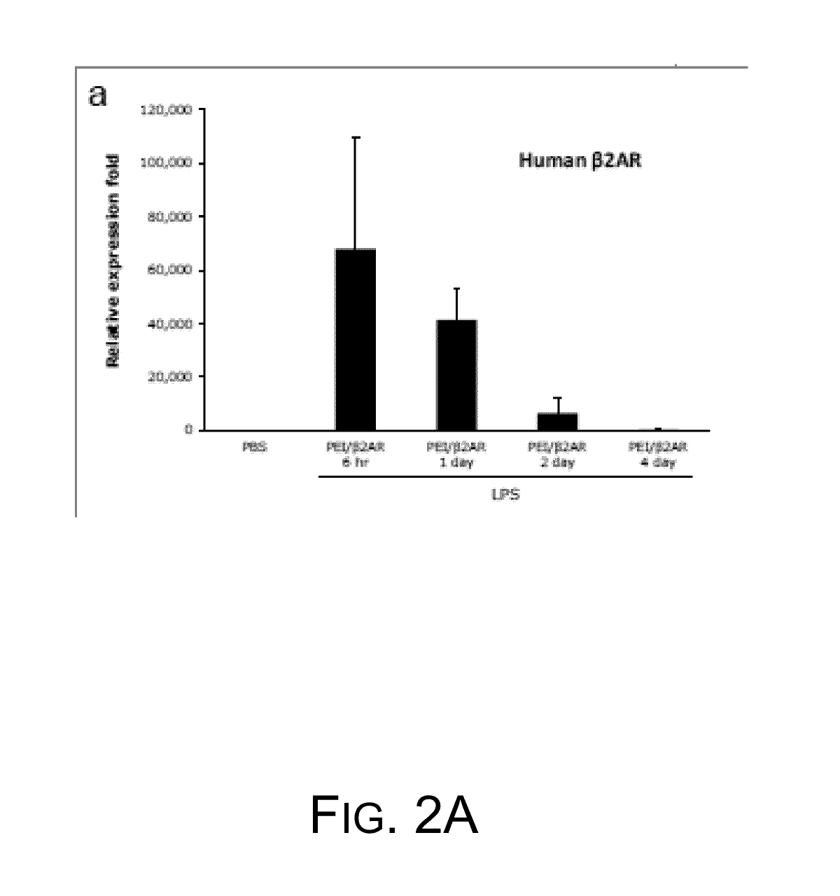

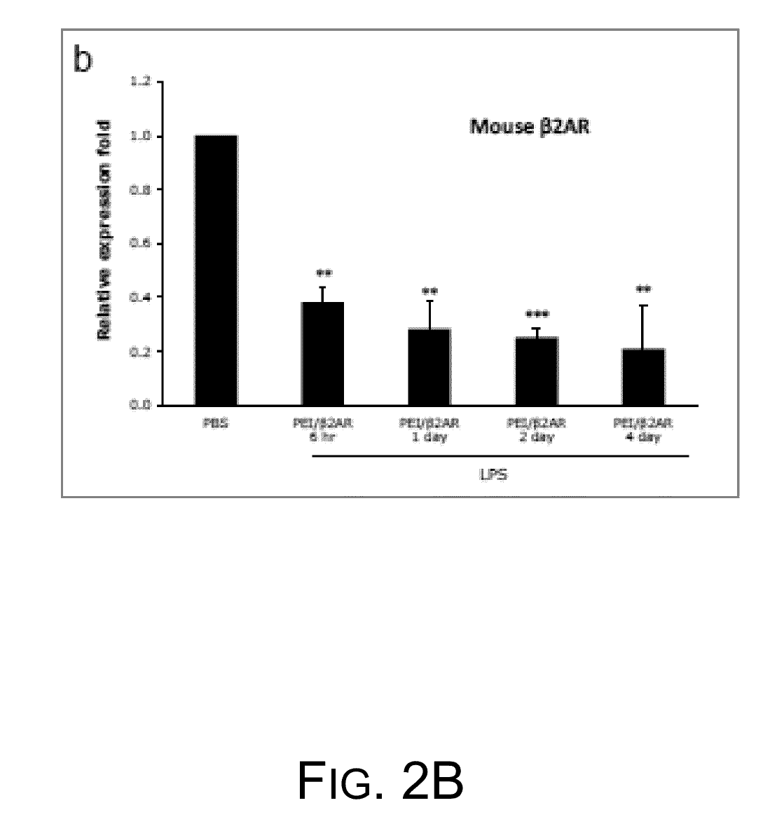

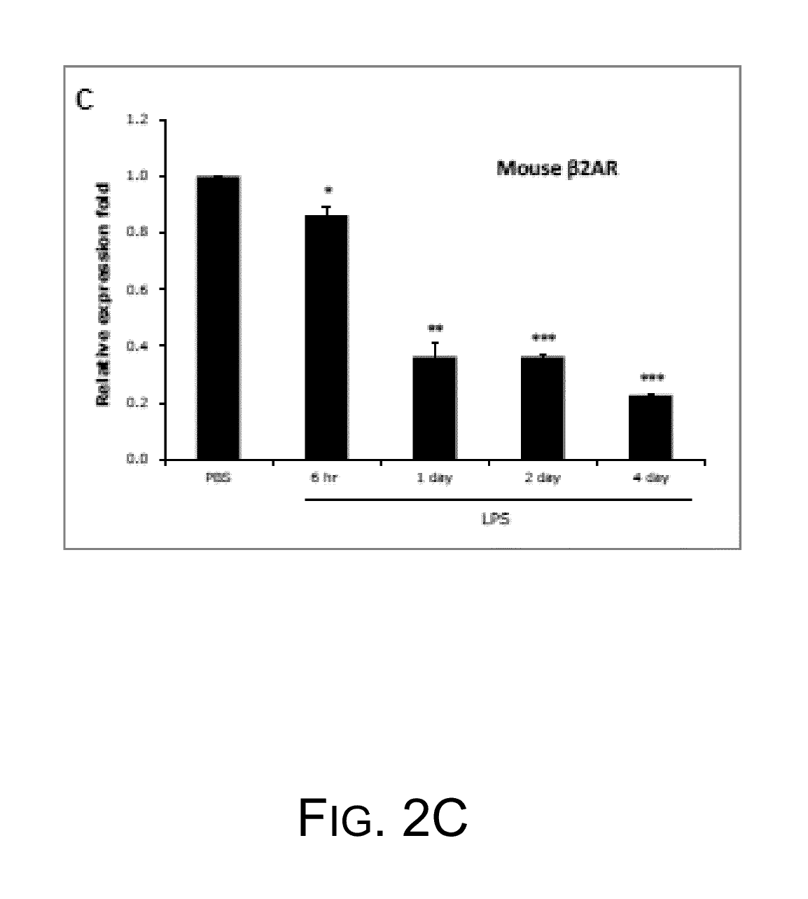

To establish the gene therapy model, human β2AR gene was then applied as the therapeutic gene for the treatment of LPS-induced ALI in the mouse model. The human β2AR gene has previously been shown to function on mice. To confirm the expression of therapeutic gene in mouse with ALI, PEI/β2AR was injected into the mice about 1 h after LPS instillation, and the mRNAs were extracted from mice lungs at indicated time points of post-transfection for Real-Time PCR analysis. As it has been reported that prolonged β2AR agonist stimulation in lung may finally cause desensitization and down-regulation of β2AR, the endogenous (mouse) β2AR expression was detected as well to identify the possible influence of exogenous β2AR delivery. The kinetics of exogenous (human) β2AR expression (FIG. 2a) was similar to that of luciferase observed in BLI study (FIG. 1a). Interestingly, at any time point after gene delivery, endogenous β2AR expression was substantially repressed (FIG. 2b). To verify whether the repression was due to ALI or exogenous β2AR overexpression, the endogenous β2AR expression in mice lungs with ALI but not been treated with PEI/β2AR was detected (FIG. 2c). The result showed that ALI itself indeed repressed endogenous β2AR expression up to 4 days of post-injury even in the absence of exogenous β2AR. This result is consistent with the previous observation that during ALI, accumulation of pulmonary edema is not only due to the increased fluid flux into airspace, but also due to the impairment of AFC mechanisms. The repression of endogenous β2AR expression observed here might be due to the impaired AFC which can limit the outcome of β2AR agonist in ALI treatment. Together, these results would suggest that exogenous β2AR delivery might be a better choice to activate β2AR downstream signaling in the treatment of ALI.

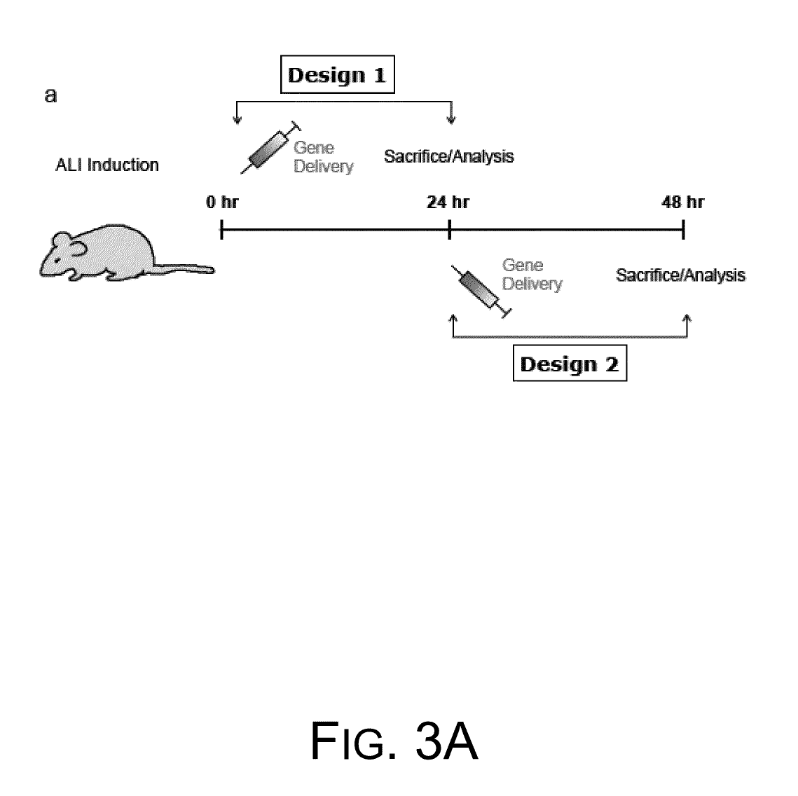

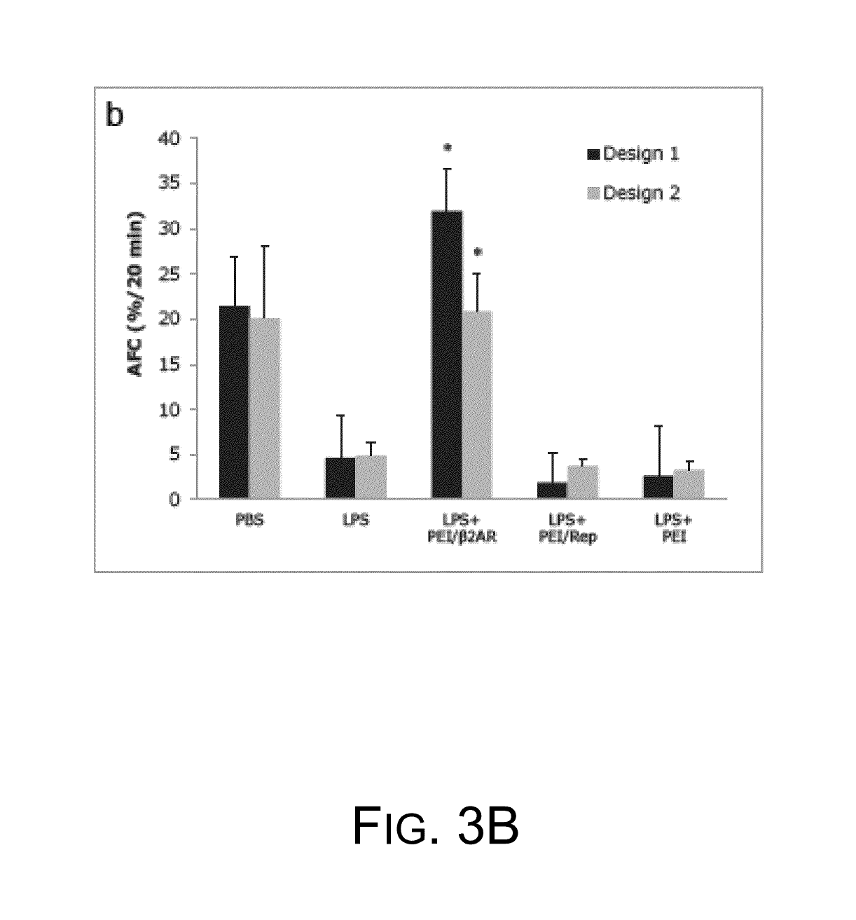

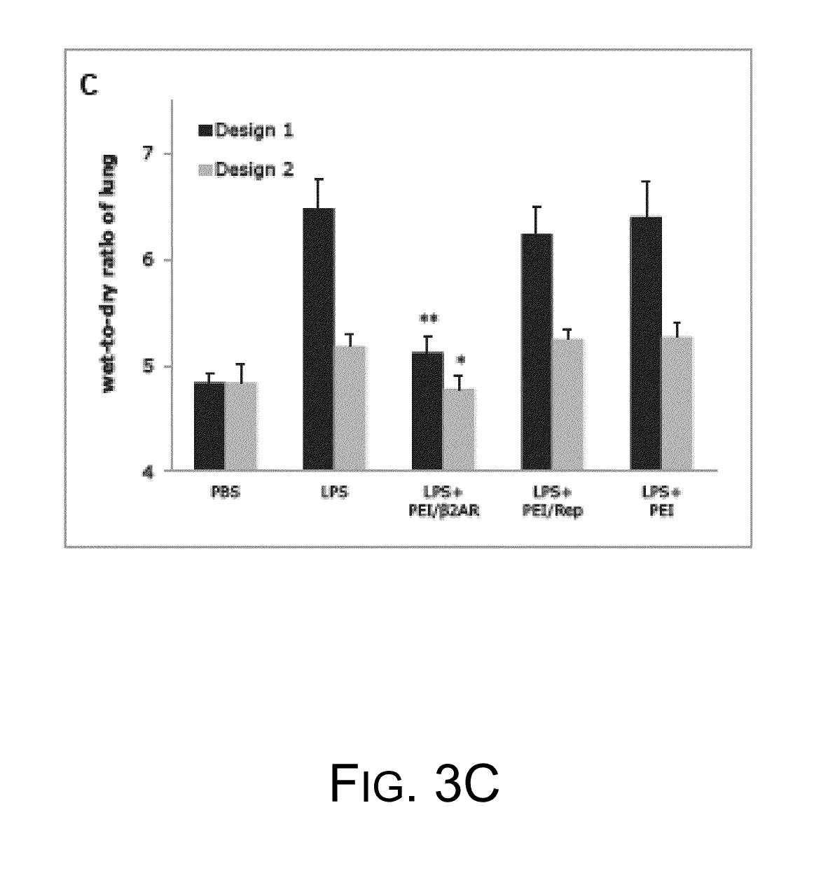

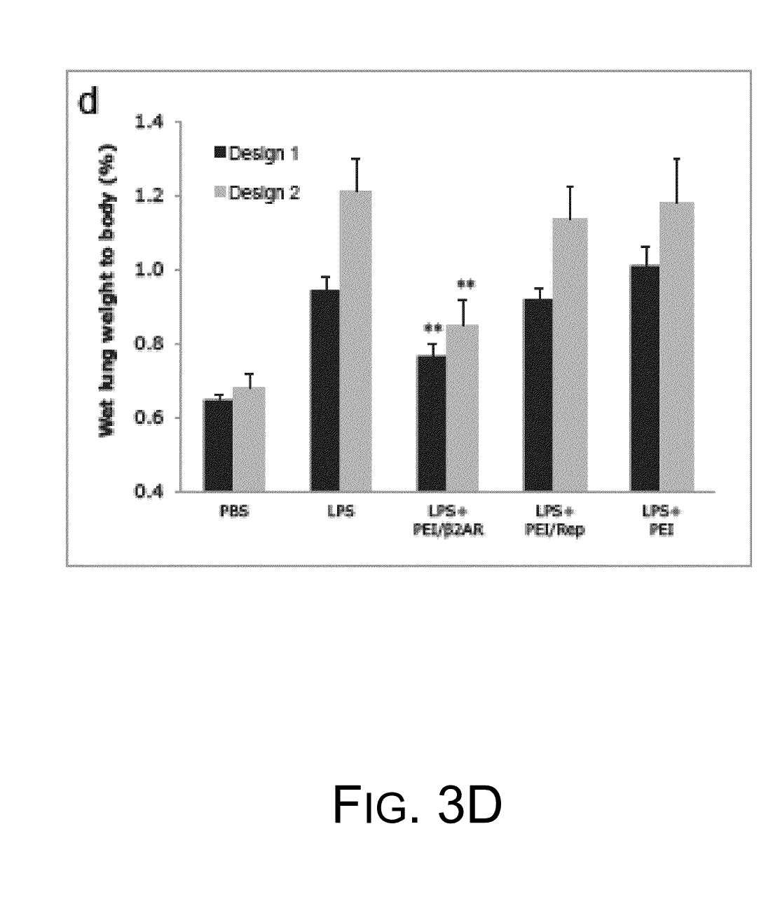

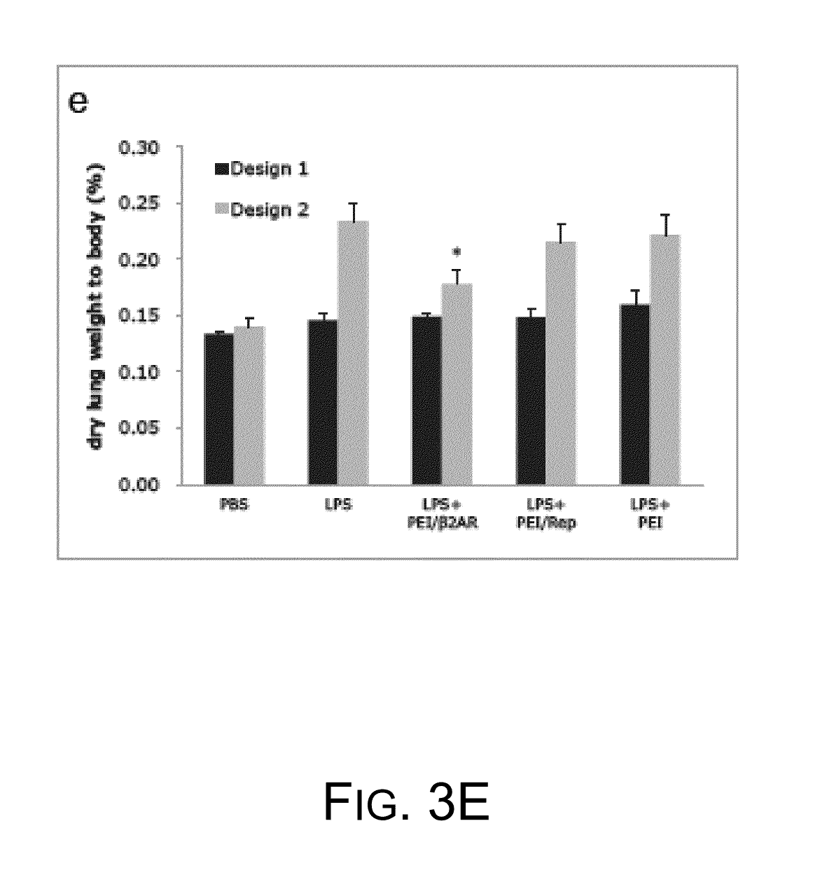

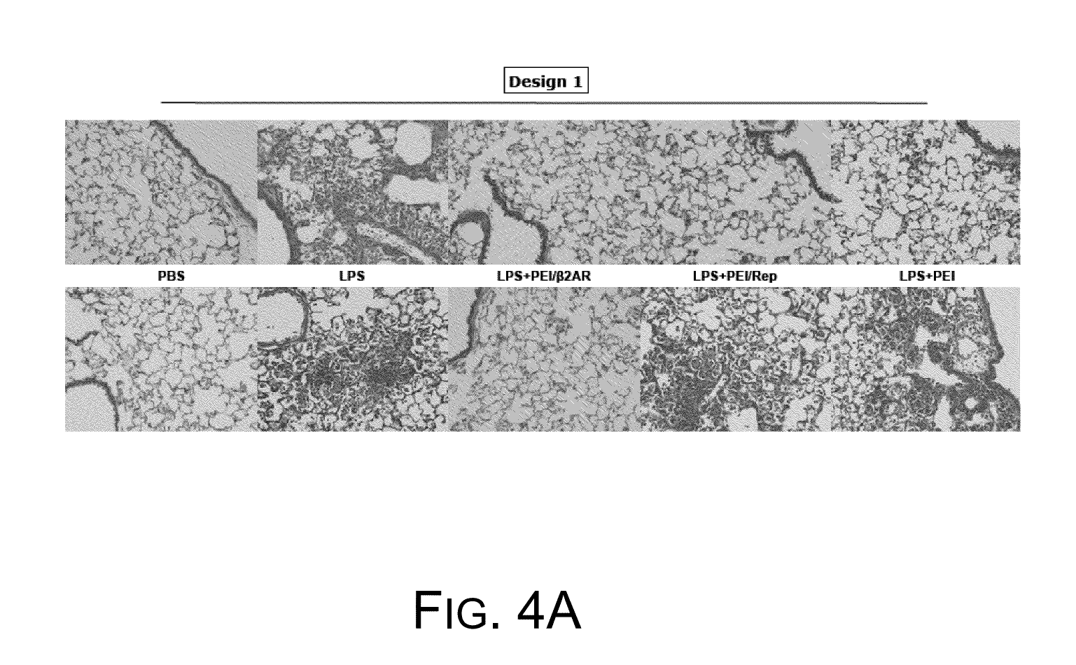

PEI/β2AR treatment was administrated in two designs: PEI/β2AR was injected at 1 hour (Design 1) or at 24 hours (Design 2) of post-injury, and the therapeutic outcomes were evaluated in 1 day after the injections (FIG. 3a). Since β2AR signaling has been reported to improve the resolution of pulmonary edema, the influence of PEI/β2AR treatment on AFC was firstly assessed. As shown in FIG. 3b, LPS-induced ALI resulted in a severe loss of AFC activity in 24 hours (Design 1) and in 48 hours (Design 2) of post-injury when comparing to non-injured mice (PBS). Treatment with PEI/β2AR in both designs significantly recovered the AFC as compared to the non-treated group (LPS). In contrast, treatment with PEI/Rep (incorporating a reporter gene, luc or lacZ) or PEI alone showed no effect in improving AFC. The level of lung water content was then analyzed by the ratio of wet lung to dry lung (wet-to-dry) weights. Consistent with the result obtained from AFC measurement, ALI induced an increase in wet-to-dry ratios in 24 hours (Design 1) and in 48 hours (Design 2) of post-injury (FIG. 3c), but PEI/β2AR treatment in both designs significantly reduced these ratios. Following the same trend, treatment with PEI/Rep or PEI alone showed no effect. It was noticed that the wet-to-dry ratio in mice with ALI decreased from 24 to 48 hours of post-injury (FIG. 3c, LPS, Design 1 and 2). The changes in wet lung and dry lung weights were studied (FIGS. 3d-3e). It was found that the wet lung weight actually continued to increase from 24 hours to 48 hours of post-injury (FIG. 3d, LPS, Design 1 and 2). It thus appeared that the increase in wet lung weight in 48 hours of post-injury was not due to the increase in water content. Indeed, the dry lung weight significantly increased from 24 hours to 48 hours of post-injury (FIG. 3e, LPS, Design 1 and 2). The weight increase in dry lung material may come from the accumulations of serum proteins or external cells in the lung. Interestingly, PEI/β2AR treatment also significantly reduced the dry lung weight in Design 2 (FIG. 3e), which could be due to reduced fluid influx after treatment. Again, the treatment with PEI/Rep or PEI alone showed no effect in both designs. These results showed that PEI-mediated β2AR gene delivery significantly improved AFC, and reduced the accumulations of lung water content and dry lung materials in mice with pre-existing ALI. These results are in consistent with the published results that β2AR overexpression can enhance AFC and reduce the lung water content, but it is the first time to show that in vivo delivery of PEI/β2AR in mice with pre-existing ALI can rapidly and effectively improve the resolution of pulmonary edema.

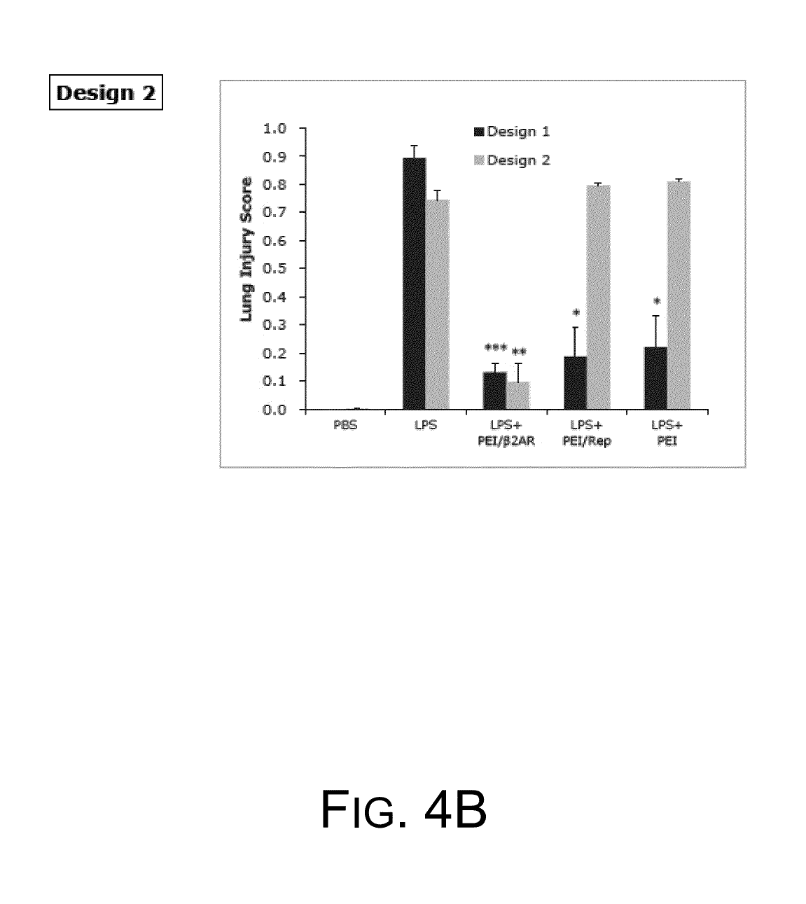

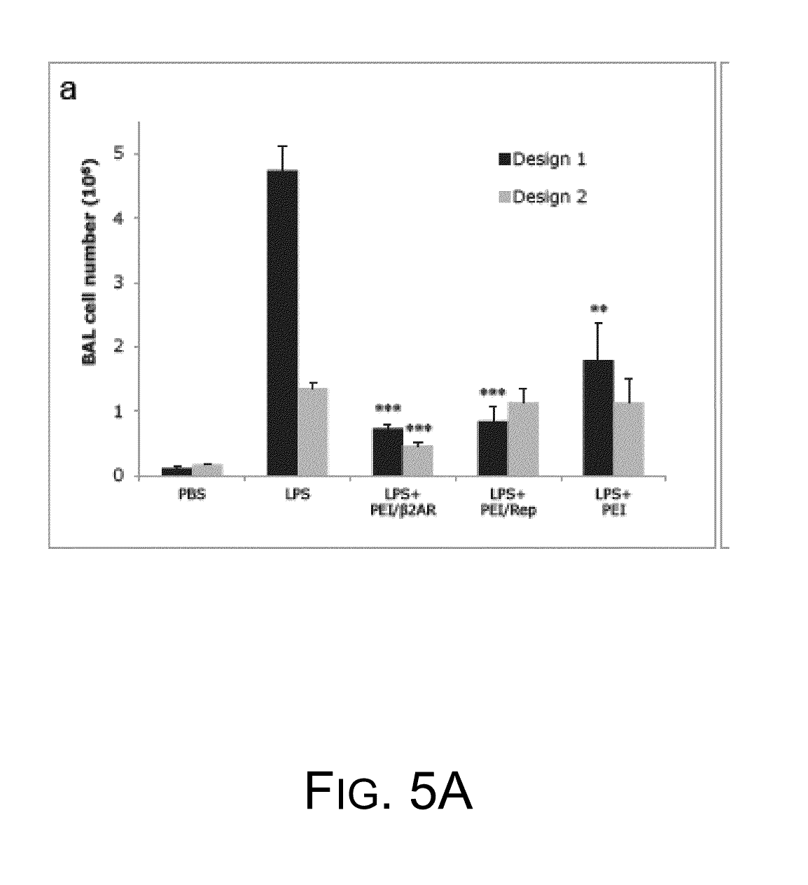

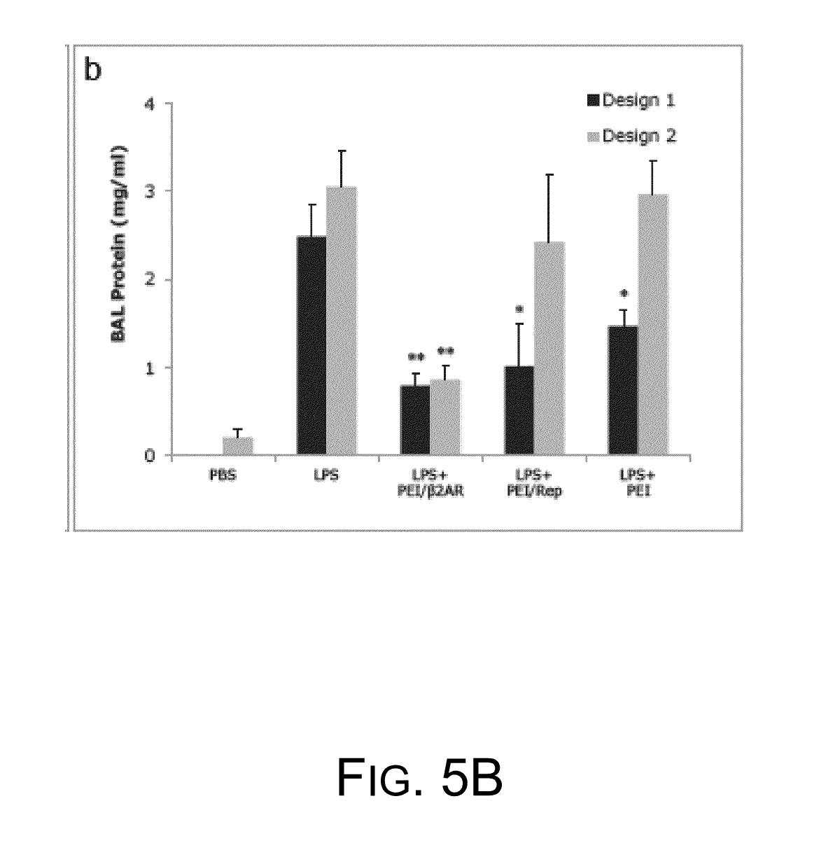

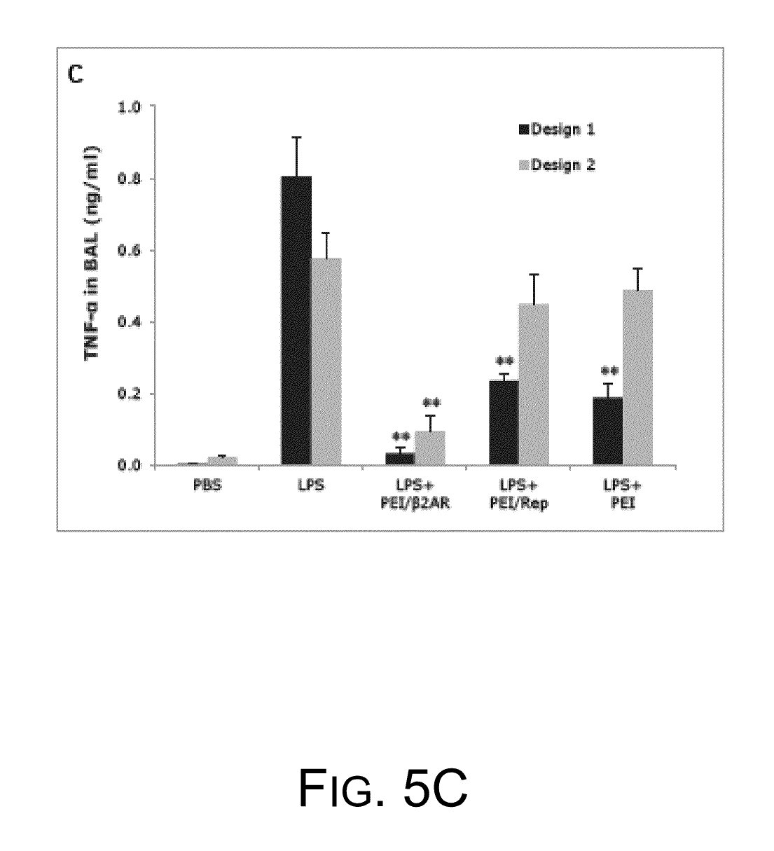

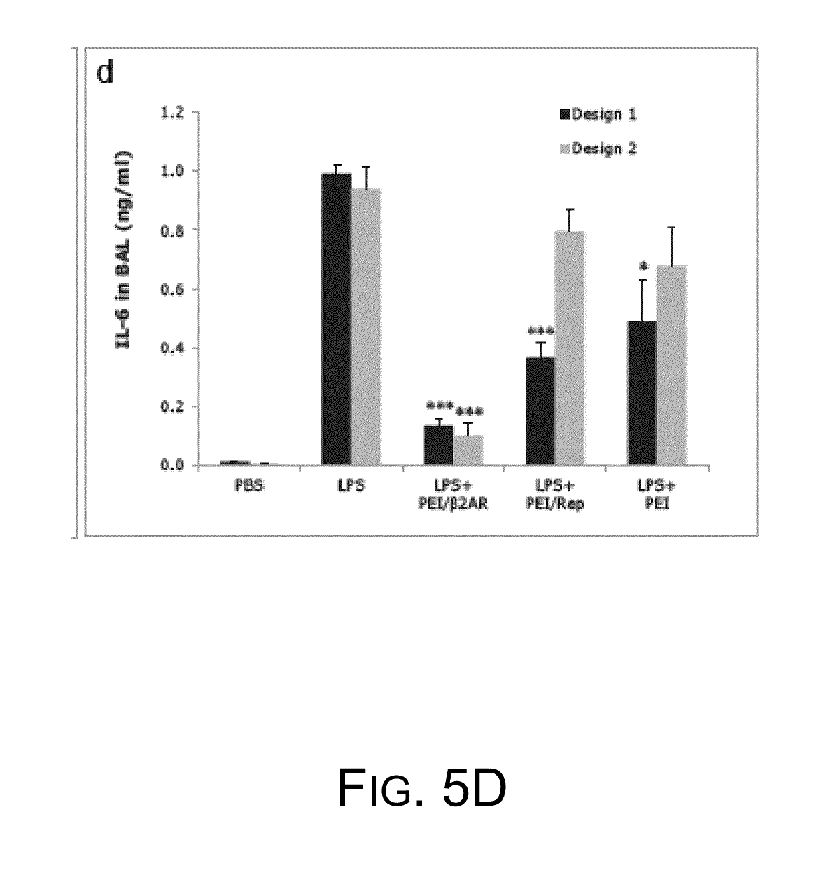

The lung histopathology was examined. As expected, LPS exposure caused extensive morphological damages including edema, hemorrhage, thickening of alveolar walls, and an increase of infiltration of neutrophils in alveolar and interstitial spaces (FIG. 4). These morphological changes were much less pronounced in lungs treated with PEI/β2AR in both designs. Unexpectedly, treatment with PEI/Rep or PEI alone in Design 1 also showed significant improvement in histopathology scores. In Design 2, however, PEI/Rep or PEI treatment showed no effect (FIG. 4). To confirm this observation, the BAL was investigated. ALI induced a dramatic increase in cell number and protein concentration in BAL, as compared to non-injured mice (PBS) (FIGS. 5a-5b). Likewise, PEI/β2AR treatment significantly decreased the levels of cell number and protein concentration in BAL in both designs. Consistent with the histopathological examination (FIG. 4), PEI/Rep or PEI treatment indeed significantly reduced the BAL cell number and protein concentration in Design 1, but not in Design 2 (FIGS. 5a-5b). The inflammatory cytokines in BAL were also detected by ELISA. As shown in FIGS. 5c-5d, ALI induced a dramatic enhancement in the levels of TNF-α and IL-6 in BAL. Following the same trend, PEI/β2AR treatment also significantly reduced the levels of these cytokines in both designs. Although not as efficient as PEI/β2AR treatment, PEI/Rep or PEI treatment still resulted in significant reductions in the cytokines in Design 1. However, in Design 2, PEI/Rep or PEI showed no statistical difference as compared to LPS group (FIGS. 5c-5d). The unexpected improvements observed in PEI/Rep or PEI treatment in Design 1 may be due to the capability of PEI nanoparticles to capture endotoxin. It is possible that in Design 1, injection of positively charged PEI or PEI/Rep nanoparticles in 1 hour of post-injury resulted in a rapid absorption/neutralization of part of instilled LPS, as a result, it alleviated the LPS-induced ALI in mice. In these experiments, two different reporter genes were applied (n=5 for luciferase and n=5 for lacZ) to confirm the effect of PEI/Rep. Since they showed similar results, two sets of data were represented as one.

As shown in FIGS. 3-5, in both designs, PEI/β2AR treatment showed significant improvements in a number of ALI criteria, including AFC, lung water content, histopathology, BAL cell number, protein level, and inflammatory cytokine levels in both designs in mice with pre-existing ALI. Although PEI/Rep or PEI treatment showed unexpected improvements in these indexes similar to PEI/β2AR treatment in Design 1, such improvements were not observed in Design 2, where PEI/Rep or PEI was administrated in 1 day of post-injury, at which time the ALI syndrome was already severe. Therefore, these results indicate that PEI/β2AR gene therapy is indeed effective in treating ALI not only when the ALI syndromes are just developed (Design 1), but also when the ALI syndromes become severe (Design 2).

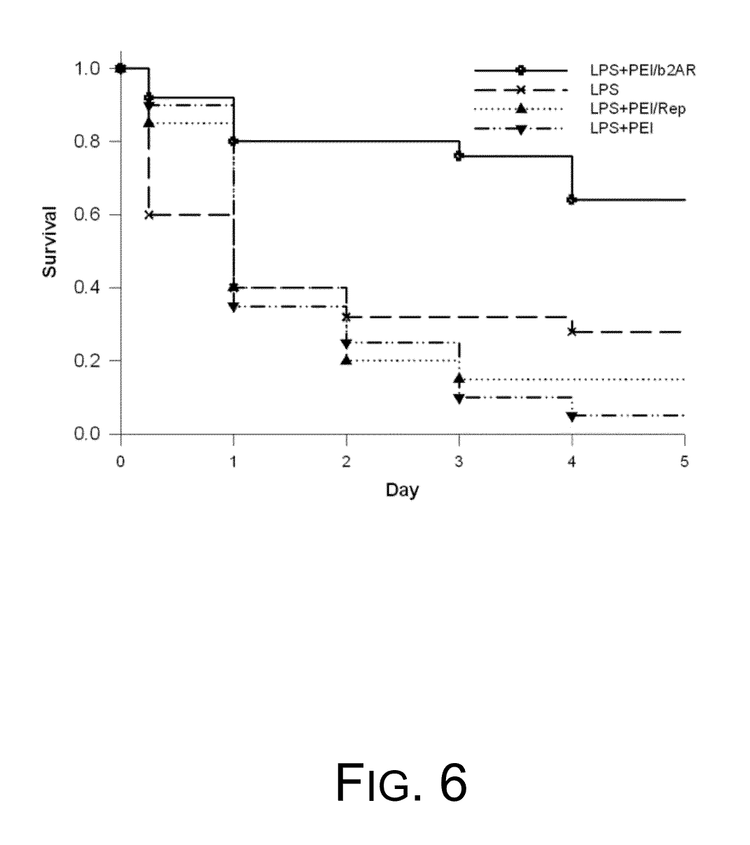

ALI/ARDS is a life-threatening syndrome with substantial mortality as high as 40% in human. The survival rate of mice with severe ALI after PEI/β2AR treatment was studied. Intratracheal administration of high-dose LPS (40 mg/kg) in mice resulted in a rapid and high mortality, with the survival rate dropped to 0.6 at 6 hours, and to 0.4 at day 1 of post-injury (FIG. 6). The injection of PEI/β2AR in mice in 1 hours of post-injury greatly improved the survival rate, which was 0.92 at 6 hours and 0.8 at day 1, respectively (FIG. 6). Surprisingly, mice treated with PEI/Rep or PEI also showed an improved survival similar to mice treated with PEI/β2AR (>0.8) at 6 hours of post-injury, but soon dropped to the level close to those LPS group after day 1. This could be attributed to the rapid absorption of part of LPS in the initial stage of treatment due to the presence of PEI. Because of the lack of a later therapeutic gene function of PEI or PEI/Rep, no survival benefit could be observed after this initial stage. No more death was observed after day 4, and the final survival rate was 0.64, 0.15, 0.05, and 0.28 in PEI/β2AR-, PEI/Rep-, PEI-, and non-treated groups, respectively. Therefore, PEI/β2AR treatment showed significant survival benefit (P<0.01) as compared to any other groups, while no statistical difference was found among the 3 other treatments.

The present disclosure provides a simple and effective gene therapy for ALI treatment. PEI-mediated β2AR delivery is rapid, safe, and transient, which improved a number of ALI criteria and the survival in mice with pre-existing ALI, without a major adverse effect observed. In particular, no additional pro-inflammatory effect other than LPS induction when using PEI/β2AR for treatment was observed.

The following examples present embodiments of the methods and compositions of the present disclosure.

### Materials and Methods

Plasmids. Luciferase and lacZ expression vectors (pT3-luc and pT3-lacZ, respectively) were kindly provided by Dr. Coll (INSERM-UJF U823, France). Additionally, pcDNA3-flag-β2AR was purchased from Addgene (#14697). Plasmids were purified using Mega-prep endotoxin-free kit (Qiagen, Hilden, Germany) and suspended in nuclease-free water, stored at −20° C. before use.

Mouse Model of ALI. Five-week-old outbred ICR mice were purchased from BioLasco Taiwan and maintained in Taiwan Mouse Clinic in Institute of Biomedical Sciences, Academia Sinica. All animal experiment protocols are approved by Academia Sinica Institutional Animal Care and Utilization Committee. For ALI induction, mice were anesthetized with Zoletil (25 mg/kg body weight) and Rompun (10 mg/kg body weight) by intraperitoneal (IP) injection. LPS (E. coli serotype 055:B5, Sigma-Aldrich, St. Louis, Mo.) suspended in PBS was intratracheally instilled into mouse lung via a 20-gauge catheter. The dose of LPS was 10 mg/kg body weight in all experiments except the survival assay, in which a dose of 40 mg/kg was administrated.

In Vivo Gene Delivery in Mouse Lung. In vivo delivery of PEI/DNA was performed following the literature procedures (Lin, E. H. et al., Biomaterials 32, 1978, 2011). Plasmid DNA was diluted in 5% glucose in a final volume of 100 μl. Linear 22-kD PEI (In vivo jetPEI, PolyPlus Transfection) was diluted in 5% glucose in a final volume of 100 μl, and added to the DNA solution. The solutions then were mixed thoroughly by a 10-sec vortex, and stood at room temperature (RT) for 15 min before being injected into lateral tail-vein in mice. The ratio of PEI to DNA is expressed as N/P ratio (the molar ratio of PEI Nitrogen to DNA Phosphate), which is maintained at 8. For therapeutic treatments (FIGS. 3-5), the dose of β2AR expression vector (pcDNA3-flag-β2AR) was uniformly 30 μg, and the PEI/DNA was injected in mice in 1 h (Design 1) or 24 h (Design 2) after LPS instillation.

Bioluminescent Imaging. The luciferase expression in living mice was analyzed by non-invasive Bioluminescent Imaging (BLI) with an IVIS-Quantum optical system (Caliper Life Sciences, Hopkinton, Mass.) following the literature procedures (Lin, E. H. et al., Biomaterials 32, 1978, 2011). Mice were injected IP with Firefly Lucirefin Potassium Salt (NanoLight Tech, Pinetop, Ariz.) dissolved in PBS (150 mg/kg body weight) before isoflurane-mediated anesthesia. Imaging was performed 5 min after luciferin injection, and the quantitative bioluminescence intensity was determined as Total Photon Flux per Second (Total Flux (p/s)) by Living Image Program™ (Caliper Life Sciences).

Real-Time PCR. Mouse lung tissue was homogenized using TissueRuptor (Qiagen) according to the manufacturer's instructions. RNA was extracted from the clear supernatant using MaestroZol Reagent (Maestrogen, Las Vegas, Nev.), quantified, and stored in RNase-free water at −80° C. until use. Reverse transcription was performed using Superscript III reverse transcriptase (Invitrogen, Grand Island, N.Y.), and the cDNAs were subjected to real-time PCR on a 96-well/plate Lightcycler 480 machine using SybrGreen MasterMix (Roche, Basel, Switzerland) for 45 cycles. Primers to amplify specific transcripts are as follows: mouse β2AR, (forward) 5′-GTACTGTGCCTAGCCTTAGCGT-3′and (reverse) 5′-GGTTAGTGTCCTGTCAAGGAGG-3′. Human β2AR, (forward) 5′-TCGCTACTTTGCCATTACTT-3′ and (reverse) 5′-CTTCCTTACGGATGAGGTTAT-3′. Mouse GAPDH, (forward) 5′-GCCTTCCGTGTTCCTAC-3′ and (reverse) 5′-CTGCTTCACCACCTTCTT-3′. Each sample was run in triplicate. The specificity of the amplification was confirmed by melt curve analysis. β2AR gene expressions were normalized by GAPDH, and the relative gene expressions were determined using the 2-ΔΔCT method.

Measurement of Alveolar Fluid Clearance (AFC) Rate in Live Mice. After irreversible anesthesia, the mouse was maintained at body temperature (37° C.), and the trachea was cannulated with a 20-gauge catheter, which was connected to a ventilator (SAR-830 Small Animal Ventilators, CWE-Inc., Ardmore, Pa.). The mouse was ventilated with a tidal volume of 0.22 ml at a frequency of 90 breaths per minute. The Evans Blue-labeled bovine serum albumin (EB-BSA) was prepared by mixing EB (0.15 mg/ml) in 5% BSA in Ringer's Lactate solution. A total of 400 μl of EB-BSA was instilled into the lung, and the mouse was ventilated for 20 minutes, after which the chest was opened to allow aspiration of fluid from the tracheal catheter. The density of EB in aspirate was measured. The AFC rate was expressed as the percentage of cleared volume in 20 minutes, calculated with the following equation: AFC (%)=100×(1−C0/C20), where C0 is the EB-BSA concentration before instillation, and C20 is the EB-BSA concentration in the aspirate at the end of 20-minute ventilation.

Measurement of Lung Water Content. After irreversible anesthesia, mice were weighed and exsanguinated by laceration on heart. Lungs were removed and wet lung weights were determined. The lungs were then incubated at 70° C. for 72 h to remove all moisture, and dry lung weights were determined. The level of lung water content was assessed by the ratio of wet lung to dry lung weight (wet-to-dry ratio).

Histological Examination. The X-gal staining, fixation, incubation, section, and Haematoxylin-Eosin (HE) staining of lung tissue were performed following the literature procedures (Lin, E. H. et al., Biomaterials, 32, 1978, 2011). Briefly, after sacrifice, mice lungs were removed, inflated and fixed by instilling 4% PFA through trachea, followed by immersion in 4% PFA at 4° C. overnight. Lungs were embedded in paraffin and sectioned at a thickness of 5 μm, and then stained with HE for microscopic examination.

For X-gal staining, Fixation buffer and X-Gal staining buffer (β-Galactosidase Reporter Gene Staining Kit, Sigma-Aldrich) were prepared according to the manufacturer's instructions. Mice transfected with lacZ were sacrificed 1 day after transfection. Lungs were removed, inflated and fixed by instilling Fixation buffer through trachea, followed by immersion in Fixation buffer for 5 min. Lungs were then washed and incubated in X-Gal staining buffer at 37° C. for 2 hours, followed by immersion in 4% PFA at 4° C. before embedded in paraffin. Ten μm-thick sections were stained with HE for microscopic examination.

Lung Injury Score. The lung injury score was assessed on histopathology. Each HE-stained sample was evaluated by 2 clinicians independently and data obtained are averaged. For each condition, 15 fields (×400 magnification, including a total of >300 alveoli) of lung sections from 3 mice were analyzed. To generate the lung injury score, points were assigned within each field according to the criteria previously addressed (Matute-Bello, G. et al., Am. J. Respir. Cell Mol. Bio. 44, 725, 2011).

Bronchioalveolar Lavage (BAL) Analysis. After irreversible anesthesia, mice were weighed and exsanguinated by laceration on heart. The BAL fluid was collected by intratracheally instilling and flushing air spaces of mouse lung with 3 ml of PBS (1 ml×3 times). Cells in BAL were collected by centrifuge (1,500 rpm, 5 min), and the supernatant was stored at −80° C. until use. BAL cell morphologies were assessed in microscope after cytospin (1,000 rpm, 5 min) and Wright-Giemsa staining. The ratio of neutrophil in each BAL sample was determined from 10 random microscopic images. Levels of TNF-α and IL-6 in BAL supernatant were measured using ELISA kit (eBioscience, San Diego, Calif.). All samples were performed in triplicate, and the spectrophotometry was determined using a VersaMax Microplate Reader (Molecular Devices, Sunnyvale, Calif.).

Statistical analyses. Two-tailed and unpaired Student's t-tests were used for statistical analyses. For survival analyses, Gehan-Breslow test was applied. P<0.05 is considered significant.

### EXAMPLES

### Example 1

### PEI-Mediated Gene Delivery to Mouse Lung Under Healthy Condition or ALI

PEI/DNA nanoparticles were injected through lateral tail-vein into healthy mice or mice with pre-existing ALI. Different amounts of reporter gene expression vectors (0 to 50 μg) were complexed with 22-kD linear PEI in a constant N/P (PEI nitrogen/DNA phosphate) molar ratio of 8. To determine the cell types targeted by PEI/DNA delivery, mice were sacrificed in 1 day after PEI/lacZ delivery, and lungs were removed and stained with X-Gal and haematoxylin and eosin (HE) for histochemical examination. 10 mg/kg of LPS was intratracheally instilled in mouse lung 1 hour prior to the PEI/DNA injection.

### Example 2

### Exogenous and Endogenous β2AR Gene Expressions in Mice Lungs with ALI

ALI was induced in mice by intratracheal instillation of 10 mg/kg of LPS. The mice were then injected with PEI/β2AR in 1 hours of post-injury. Mice were sacrificed at indicated time points of post-transfection, and the lungs were harvested for RNA extraction. PBS was instilled into mice lungs as non-injury control (PBS). Exogenous (human) (2a) and endogenous (mouse) β2AR gene expressions were detected using Real-Time PCR with specific primers, and presented as ratios relative to PBS group.

### Example 3

### PEI/β2AR Treatment on Mice with Pre-Existing ALI

PEI/β2AR was induced in mice by intratracheal instillation of 10 mg/kg of LPS, and the gene therapy was administrated in two designs. In Design 1, mice were injected with PEI/β2AR at 1 hours of post-injury and sacrificed at 24 hours; while in Design 2, mice were injected with PEI/β2AR at 24 hours of post-injury and sacrificed at 48 hours, for evaluation of therapeutic outcome. AFC was measured in vivo. Lung water content was assessed by the measurement of wet-to-dry ratio. For control, mice were treated with reporter genes (LPS+PEI/Rep, with half of luciferase and half of lacZ), or PEI alone (LPS+PEI). PBS was instilled into mice lungs as non-injury control (PBS). The PEI/DNA complex uniformly contained 30 μg of DNA with an N/P ratio of 8.

### Example 5

### PEI/β2AR Treatment Improved the Survival of Mice from Severe ALI

For survival assay, mice were intratracheally instilled with a high-dose LPS (40 mg/kg), and then treated with PEI/β2AR at 1 hours of post-injury. For control, mice were treated with reporter genes (LPS+PEI/Rep), or PEI alone (LPS+PEI). The survival of mice was recorded after 6 hours of post-injury.

While the preferred embodiment of the present disclosure has been illustrated and described, it will be appreciated that various changes can be made therein without departing from the spirit and scope of the present disclosure.

1. A method of delivering β2-Adrenergic Receptor (β2AR) genes to a lung of a patient in need thereof to treat a lung disease in the patient, the method comprising:
administering to the patient a polyplex comprising a linear polyethyleneimine (PEI) and a DNA comprising the β2AR gene.

2. The method as recited in claim 1, wherein the lung disease comprises acute lung injury and acute respiratory distress syndrome.

3. The method as recited in claim 1, wherein the linear PEI has a molecular weight of about 22,000 to about 25,000 daltons.

4. The method as recited in claim 1, wherein a molar ratio of nitrogen atoms in the linear PEI to phosphates in the DNA comprising the β2AR gene is about 6 to 10.

5. The method as recited in claim 1, wherein a weight ratio of the DNA comprising the β2AR gene to the patient is less than 2.5×10⁻⁶.

6. The method as recited in claim 1, wherein the administering comprises administering via intravenous injection.

7. A method of delivering β2-Adrenergic Receptor (β2AR) genes into alveolar epithelial cells of a lung of a patient in need thereof to treat acute lung injury in the patient, the method comprising:
mixing a linear polyethyleneimine (PEI) with a DNA comprising the β2AR gene to form a polyplex; and
administering the polyplex to the alveolar epithelial cells of the lung via intravenous injection.

8. The method as recited in claim 7, wherein the linear PEI has a molecular weight of about 22,000 to about 25,000 daltons.

9. The method as recited in claim 7, wherein a molar ratio of nitrogen atoms in the linear PEI to phosphates in the DNA comprising the β2AR gene is about 6 to about 10,.

10. The method as recited in claim 7, wherein a weight ratio of the DNA comprising the β2AR gene to the patient is less than 2.5×10⁻⁶.

11. A polyplex for delivering a β2-Adrenergic Receptor (β2AR) gene to a lung of a patient in need thereof, comprising:
a linear polyethyleneimine (PEI) and a DNA comprising the β2AR gene,
wherein the polyplex is effective to treat symptoms of a pre-existing acute lung disease in the patient.

12. The polyplex as recited in claim 11, wherein the linear PEI has a molecular weight of about 22,000 to about 25,000 daltons.

13. The polyplex as recited in claim 11, wherein a molar ratio of nitrogen atoms in the linear PEI to phosphates in the DNA comprising the β2AR gene is about 8.

14. The polyplex as recited in claim 11, wherein a mass ratio between the DNA comprising the β2AR gene and the patient is less than 2.5×10⁻⁶.

15. The polyplex as recited in claim 11, wherein the pre-existing acute lung disease comprises acute lung injury and acute respiratory distress syndrome.


SEQUENCE LISTING (9 sequences)

SEQ ID NO: 1 - DNA, 1653 bp, Artificial sequence
  A reporter gene
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 2 - DNA, 3144 bp, Artificial sequence
  A reporter gene
atgtcgtttactttgaccaacaagaacgtgattttcgttgccggtctgggaggcattggtctggacaccagcaaggagctgctcaagcgcgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattacagctgagcgccggtcgctaccattaccagttggtctggtgtcaaaaataa

SEQ ID NO: 3 - DNA, 1242 bp, Homo sapiens
atggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaa

SEQ ID NO: 4 - DNA, 22 bp, Artificial sequence
  Primer
gtactgtgcctagccttagcgt

SEQ ID NO: 5 - DNA, 22 bp, Artificial sequence
  Primer
ggttagtgtcctgtcaaggagg

SEQ ID NO: 6 - DNA, 20 bp, Artificial sequence
  Primer
tcgctactttgccattactt

SEQ ID NO: 7 - DNA, 21 bp, Artificial sequence
  Primer
cttccttacggatgaggttat

SEQ ID NO: 8 - DNA, 17 bp, Artificial sequence
  Primer
gccttccgtgttcctac

SEQ ID NO: 9 - DNA, 18 bp, Artificial sequence
  Primer
ctgcttcaccaccttctt